(19) United States
(12) Patent Application Publication
(10) Pub. No.: US 2024/0287084 A1
(43) Pub. Date: Aug. 29, 2024

(54) SYNTHESIS OF [1,2,3]TRIAZOLO[4,5-D]PYRIMIDINES

(71) Applicant: Hoffmann-La Roche Inc., Little Falls, NJ (US)

(72) Inventors: Jean-Michel Adam, Rosenau (FR); Uwe Grether, Efringen-Kirchen (DE); Christian Moessner, Lörrach (DE); Paolo Tosatti, Binningen (CH)

(73) Assignee: Hoffmann-La Roche Inc., Little Falls, NJ (US)

(21) Appl. No.: 18/644,781

(22) Filed: Apr. 24, 2024

Related U.S. Application Data
(63) Continuation of application No. PCT/EP2022/079854, filed on Oct. 26, 2022.

(30) Foreign Application Priority Data
Oct. 28, 2021 (EP) 21205140.3
Feb. 9, 2022 (EP) 22155794.5

(51) Int. Cl. C07D 487/04

(52) U.S. Cl. CPC C07D 487/04

(57) ABSTRACT

The present invention relates to a process for the preparation of [1,2,3]triazolo[4,5-d]pyrimidine derivatives useful as pharmaceutically active compounds, in particular 1-[5-tert-butyl-3-[(1-methyltetrazol-5-yl)methyl]triazolo[4,5-d]pyrimidin-7-yl]pyrrolidin-3-ol.

The present invention relates to a process for the preparation of [1,2,3]triazolo[4,5-d]pyrimidine derivatives useful as pharmaceutically active compounds, in particular 1-[5-tert-butyl-3-[(1-methyltetrazol-5-yl)methyl]triazolo[4,5-d]pyrimidin-7-yl]pyrrolidin-3-ol.

The class of compounds disclosed in WO2013/068306 have shown activities as CB2 receptor agonist. The interest in CB2 receptor agonists has been steadily on the rise during the last decade (currently 30-40 patent applications/year) due to the fact that several of the early compounds have been shown to have beneficial effects in pre-clinical models for a number of human diseases including chronic pain (Beltramo, M. Mini Rev Med Chem 2009, 9(1), 11-25), atherosclerosis (Mach, F. et al. J Neuroendocrinol 2008, 20 Suppl 1, 53-7), regulation of bone mass (Bab, I. et al. Br J Pharmacol 2008, 153(2), 182-8), neuroinflammation (Cabral, G. A. et al. J Leukoc Biol 2005, 78(6), 1192-7), ischemia/reperfusion injury (Pacher, P. et al. Br J Pharmacol 2008, 153(2), 252-62), systemic fibrosis (Akhmetshina, A. et al. Arthritis Rheum 2009, 60(4), 1129-36; Garcia-Gonzalez, E. et al. Rheumatology (Oxford) 2009, 48(9), 1050-6), liver fibrosis (Julien, B. et al. Gastroenterology 2005, 128(3), 742-55; Munoz-Luque, J. et al. J Pharmacol Exp Ther 2008, 324(2), 475-83).

The present invention provides a process for the preparation of compound of formula (I) or a pharmaceutically acceptable salt:

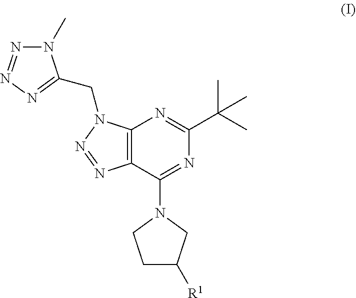

wherein
R¹ is halogen, —OH, —NR^{a}R^{b}, (C₁-C₆)alkoxy, (C₁-C₆)alkyl, —O(O)CR^{c} or —NR^{a}C(O)R^{c}, in particular R¹ is —OH;
R^{a} and R^{b} are chosen independently from H, (C₁-C₆)alkyl, (C₁-C₆) alkoxy, halo(C₁-C₆)alkyl, phenyl, halo-phenyl or (C₁-C₆)alkyl-phenyl;
R^{c} is chosen independently from H, (C₁-C₆)alkyl, (C₁-C₆)alkoxy, —OH, C₁₋₆ halo-(C₁-C₆)alkyl, phenyl, halo-phenyl or (C₁-C₆)alkyl-phenyl;
which comprises reacting a compound of formula (II), salts thereof, a tautomer thereof or a mixture of tautomers thereof

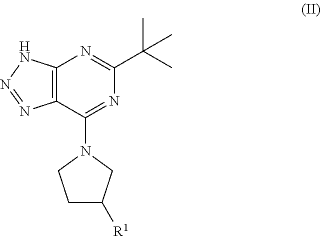

with a compound of formula (III):

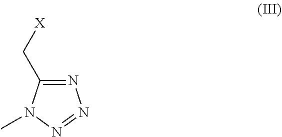

wherein
X is halogen, triflate or tosyl;
in the presence of an organic acid.

### BRIEF DESCRIPTION OF THE FIGURES

FIG. 1 illustrates a IR spectrum of 1-[5-tert-butyl-3-[(1-methyltetrazol-5-yl)methyl]triazolo[4,5-d]pyrimidin-7-yl]pyrrolidin-3-ol crystalline form, also known as Form A.

FIG. 2 illustrates a Raman spectrum of 1-[5-tert-butyl-3-[(1-methyltetrazol-5-yl)methyl]triazolo[4,5-d]pyrimidin-7-yl]pyrrolidin-3-ol crystalline form, also known as Form A.

FIG. 3 illustrates a IR spectrum of 1-[5-tert-butyl-3-[(1-methyltetrazol-5-yl)methyl]triazolo[4,5-d]pyrimidin-7-yl]pyrrolidin-3-ol amorphous form.

FIG. 4 illustrates a Raman spectrum of 1-[5-tert-butyl-3-[(1-methyltetrazol-5-yl)methyl]triazolo[4,5-d]pyrimidin-7-yl]pyrrolidin-3-ol amorphous form.

FIG. 5 illustrates a x-ray powder diffraction spectrum of 1-[5-tert-butyl-3-[(1-methyltetrazol-5-yl)methyl]triazolo[4,5-d]pyrimidin-7-yl]pyrrolidin-3-ol amorphous form.

FIG. 6 illustrates a x-ray powder diffraction spectrum of 1-[5-tert-butyl-3-[(1-methyltetrazol-5-yl)methyl]triazolo[4,5-d]pyrimidin-7-yl]pyrrolidin-3-ol crystalline form, also known as Form A.

Unless otherwise stated, the following terms used in the specification and claims have the meanings given below:

The term “(C₁-C₆)alkyl”, alone or in combination, signifies a straight-chain or branched-chain alkyl group with 1 to 6 carbon atoms, particularly a straight or branched-chain alkyl group with 1 to 6 carbon atoms and more particularly a straight or branched-chain alkyl group with 1 to 4 carbon atoms. Examples of straight-chain and branched-chain C₁-C₆ alkyl groups are methyl, ethyl, propyl, isopropyl, butyl, isobutyl, tert.-butyl, the isomeric pentyls, the isomeric hexyls, particularly methyl, ethyl, propyl, butyl and pentyl more particularly methyl, ethyl, propyl, isopropyl, isobutyl, tert.-butyl and isopentyl. Particular examples of alkyl are methyl, ethyl and pentyl, in particular methyl and ethyl.

“Compound of formula (I′)” refers to:

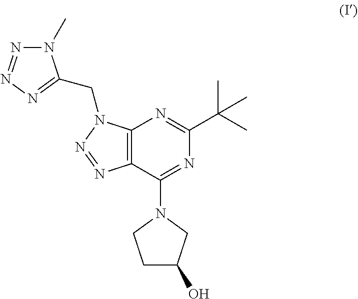 [SMILES: Cn1nnnc1Cn1nnc2c(N3CC[C@H](O)C3)nc(C(C)(C)C)nc21]

Compound of formula (I′) is also known as 1-[5-tert-butyl-3-[(1-methyltetrazol-5-yl)methyl]triazolo[4,5-d]pyrimidin-7-yl]pyrrolidin-3-ol. Herein compound of formula (I′)'s name or reference can be interchangeably used.

“Form A” as used herein refers to the crystalline polymorphic Form A of 1-[5-tert-butyl-3-[(1-methyltetrazol-5-yl)methyl]triazolo[4,5-d]pyrimidin-7-yl]pyrrolidin-3-ol.

“XRPD” refers the analytical method of X-Ray Powder Diffraction. The repeatability of the angular values is in the range of 2Theta 0.2°. The term “approximately” given in combination with an angular value denotes the repeatability which is in the range of 2Theta ±0.2°. The relative XRPD peak intensity is dependent upon many factors such as structure factor, temperature factor, crystallinity, polarization factor, multiplicity, and Lorentz factor. Relative intensities may vary considerably from one measurement to another due to preferred orientation effects. According to USP 941 (US Pharmacopoeia, 37th Edition, General Chapter 941), relative intensities between two samples of the same material may vary considerably due to “preferred orientation” effects. Anisotropic materials adopting preferred orientation will lead to anisotropic distribution of properties such as modulus, strength, ductility, toughness, electrical conductivity, thermal expansion, etc., as described e.g. in Kocks U. F. et al. (Texture and Anisotropy: Preferred Orientations in Polycrystals and Their Effect on Materials Properties, Cambridge University Press, 2000). In XRPD but also Raman spectroscopy, preferred orientations cause a change in the intensity distribution. Preferred orientation effects are particularly pronounced with crystalline APIs of relatively large particle size.

“characteristic peak” refers to the presence of the powder X-ray diffraction peak definitively identifies the 1-[5-tert-butyl-3-[(1-methyltetrazol-5-yl)methyl]triazolo[4,5-d]pyrimidin-7-yl]pyrrolidin-3-ol as the referenced crystalline form (Form A). Typically, the powder X-ray diffraction analysis is conducted at ambient conditions in transmission geometry with a STOE STADI P diffractometer (Cu Kai radiation, primary monochromator, silicon strip detector, angular range 3 to 42 degrees two-theta, approximately 30 minutes total measurement time). The samples (approximately 10 to 50 mg) are prepared between thin polymer films and are analyzed without further processing (e.g. grinding or sieving) of the substance.

“Polymorph” refers to crystalline forms having the same chemical composition but different spatial arrangements of the molecules, atoms, and/or ions forming the crystal. In general, reference throughout this specification will be to a polymorph 1-[5-tert-butyl-3-[(1-methyltetrazol-5-yl)methyl]triazolo[4,5-d]pyrimidin-7-yl]pyrrolidin-3-ol.

The term “halo-(C₁-C₆)-alkyl” means (C₁-C₆)alkyl substituted by one or more, preferably one to five, “halo” atoms, as such terms are defined in this Application. Halo(C₁-C₆)alkyl includes monohalo(C₁-C₆)alkyl, dihalo(C₁-C₆)alkyl, trihalo(C₁-C₆)alkyl, perhalo(C₁-C₆)alkyl and the like e.g. chloromethyl, dichloromethyl, difluoromethyl, trifluoromethyl, 2,2,2-trifluoroethyl, perfluoroethyl, 2,2,2-trifluoro-1,1-dichloroethyl, and the like).

The term “halo-phenyl” means halogen-substituted phenyl wherein the halogen is selected from chlorine, bromine, iodine, and fluorine.

The term “(C₁-C₆)alkyl-phenyl” means a straight or branched hydrocarbon having from 1 to 6 carbon atoms as defined above attached to a phenyl or substituted phenyl group.

The terms “halogen” or “halo”, alone or in combination, signifies fluorine, chlorine, bromine or iodine and particularly iodine, chlorine or bromine, more particularly iodine and chlorine. The term “halo”, in combination with another group, denotes the substitution of said group with at least one halogen, particularly substituted with one to five halogens, particularly one to four halogens, i.e. one, two, three or four halogens. Particular halogens are iodine, bromine and chlorine, more particularly iodine and chlorine.

The term “tautomer” means constitutional isomers that undergo such rapid interconversion that they cannot be independently isolated.

The term “phase transfer catalyst” means a compound, which is capable of transferring a water-soluble anion into an organic phase. Phase transfer catalysts comprise tetralkylammonium salts, phosphonium salts, and crown ethers. Examples of phase transfer catalysts comprise tetrasubstituted ammonium salts and trisubstituted ammonium salts, which may form tetrasubstituted ammonium salts in situ. Tetrasubstituted ammonium salts comprise tetrabutylammonium, benzyl trimethylammonium, tetraethylammonium, cetyltrimethylammonium salts in which the counter ion can be fluorine, chlorine, bromine or iodine. Trisubstituted amines comprise triethylamine, tributylamine, benzyldiethylamine, and diisopropylethylamine.

The term “inorganic base” signifies alkali base, such as alkali carbonate, alkali bicarbonate, alkali borate, alkali phosphate, alkali-hydroxide. A more preferred basic aqueous solution is chosen from solution of sodium carbonate, potassium carbonate, lithium carbonate, lithium hydroxide, potassium hydroxide, sodium hydroxide, sodium hydrogen carbonate, potassium hydrogen carbonate, or lithium hydrogen carbonate, particularly sodium hydroxide, potassium hydroxide, and lithium hydroxide, more particularly sodium hydroxide, sodium borate, or a mixture thereof. The most preferred basic aqueous solution is a solution of sodium bicarbonate, sodium hydroxide or a mixture thereof.

The term “Heterogeneous transition metal hydrogenation catalyst” refers to a transition metal hydrogenation catalyst which acts in different phase than the substrate. Especially the transition metal hydrogenation catalyst is in the solid phase. In particular while the transition metal hydrogenation catalyst is in the solid phase the reactants are in the liquid phase. The transition metal hydrogenation catalyst contains a transition metal which forms one or more stable ions which have incompletely filled d orbitals (i.e. Pd, Pt, Rh, Au, Ni, Co, Ru, Ir) in particular noble metal, such as Pd, Pt, Rh or Au. In these catalysts the transition metal is in particular “supported”, which means that the catalyst is dispersed on a second material that enhances the effectiveness. The “support” can be merely a surface on which the metal is spread to increase the surface area. The supports are porous materials with a high surface area, most commonly alumina or various kinds of carbon. Further examples of supports include, but are not limited to, silicon dioxide, titanium dioxide, calcium carbonate, barium sulfate, diatomaceous earth and clay. The metal itself can also act as a support, if no other support is present. More specifically the term “heterogeneous transition metal hydrogenation catalyst”includes but is not limited to, a Raney catalyst (e.g. Ra-Ni, Ra-Co,) Pd/C, Pd(OH)2/C, Au/TiO2, Rh/C, Ru/Al2O3, Ir/CaCO3, or Pt/C.

The term “salt” denotes those salts formed with inorganic acids such as hydrochloric acid, hydrobromic acid, sulfuric acid, carbonic acid, formic acid, acetic acid, phosphoric acid, and organic acids selected from aliphatic, cycloaliphatic, aromatic, araliphatic, heterocyclic, carboxylic, and sulfonic classes of organic acids such as methanesulfonic acid, ethanesulfonic acid, and p-toluenesulfonic acid, in particular salt refers to salts formed with hydrochloric acid and citric acid.

The terms “hydroxyl” and “hydroxy”, alone or in combination, signify the —OH group.

The term “(C₁-C₆)alkoxy”, alone or in combination, signifies a group of the formula (C₁-C₆)alkyl-O—in which the term “(C₁-C₆)alkyl” has the previously given significance, such as methoxy, ethoxy, n-propoxy, isopropoxy, n-butoxy, isobutoxy, sec.butoxy and tert.butoxy, particularly methoxy.

The term “acidic organic solution” means a solution of a solvent with an organic acid which has a pH between 1 and 4, particularly between 2 and 3, and more particularly around 2.5. Solution pH is measured by hydrogen ion content (H⁺).

The term “around” means±5%, when referred to pH ranges, means±0.1.

The term “inorganic acid” means an inorganic compound capable of giving proton of Broensted's definition, dissociating into proton and counter ion in water at 25° C. and giving a solution having neutral pH or below. Concrete examples of the inorganic acid are phosphoric acid (orthophosphoric acid), sulfuric acid, nitric acid, phosphinic acid, phosphonic acid, diphosphonic acid, hydrochloric acid, pyrophosphoric acid, metaphosphoric acid and nitrous acid. These acids may be used in the form of metal salts, ammonium salts or the like; particularly inorganic acid means hydrochloric acid.

The term “workup” means the work of isolation and/or purification which is carried out once the reaction is finished, this process can comprise a treatment of the reaction mixture with a base or acid solution, the addition of a solvent for means of extraction or precipitation of certain compounds, processes of filtration, distillation, extraction, recrystallization, or precipitation. In particular “workup” means treatment of the reaction mixture with an acidic organic solution.

The term “organic acid” means an acid, i.e. a compound that is capable of releasing a cation or proton H⁺or H₃O⁺, in aqueous medium, which comprises at least one (optionally unsaturated) linear or branched C₁-C₂₀ hydrocarbon-based chain, or a (hetero)cycloalkyl or (hetero)aryl group and at least one acid chemical function chosen in particular from carboxyl COOH, sulfonyl SO₃H, sulfinyl SO₂H, and phosphoric PO₃H₂, in particular “organic acid” refers to, lactic acid, formic acid, citric acid, oxalic acid, malic acid, and tartaric acid, particularly acetic and citric acid, more particularly citric acid.

In particular, the preparation of compound of formula I is being carried out in the presence of a biphasic solvent mixture, an inorganic base, and a phase transfer catalyst, and in the presence of an acidic organic solution during the workup;

wherein the acidic organic solution is an organic acid, particularly in a solution, more particularly selected fromlactic acid, acetic acid, formic acid, citric acid, oxalic acid, uric acid, malic acid, and tartaric acid, particularly citric acid and acetic acid, more particularly citric acid, and wherein the acidic organic solution has a pH between 1 and 4, particularly between 2 to 3, more particularly around 2.5, and wherein the concentration of the organic acid in solution is between 1% and 30%, particularly 5% and 20%, more particularly around 10%.

In some of the embodiments, the organic acid is in a suitable solvent, forming an acidic organic solution; in particular the suitable solvent is, but not limited to, water, methanol, or ethanol, particularly water.

In a more particular embodiment, the present invention provides a process as described above for the preparation of compound of formula (I) or (I′) wherein the biphasic solvent mixture is between water and any of the solvents selected from ethyl acetate, diethyl carbonate, diethyl ether, methyl t-butyl ether, isopropyl acetate, n-propyl acetate, tetrahydrofuran, MeTHF or a combination thereof is used, particularly water and ethyl acetate, n-propyl acetate, isopropyl acetate, diethyl carbonate or a combination thereof more particularly water and n-propyl acetate.

In a more particular embodiment, the present invention provides a process as described above for the preparation of compound of formula (I) or (I′) wherein the inorganic base is sodium carbonate, potassium carbonate, lithium carbonate, lithium hydroxide, potassium hydroxide, sodium hydroxide, sodium hydrogen carbonate, potassium hydrogen carbonate, or lithium hydrogen carbonate, particularly sodium hydroxide, potassium hydroxide, and lithium hydroxide, more particularly sodium hydroxide.

In a more particular embodiment, the present invention provides a process as described above for the preparation of compound of formula (I) or (I′) wherein the phase transfer catalyst is selected from a quatemary ammonium salt, an organic phosphonium salt or a crown ether, in particular tetrasubstituted ammonium salts comprise tetrabutylammonium, benzyl trimethylammonium, benzyltriethylammonium, ethyltributylammonium, methyltrioctylammonium, methyltributylammonium, propyltributylammonium, methyltricaprylammonium, tetraethylammonium, cetyltrimethylammonium salts in which the counter ion can be fluorine, chlorine, bromine or iodine, more particularly tetrabutylammonium iodide, tetrabutylammonium bromide, tetrabutylammonium chloride, most particularly, tetrabutylammonium iodide.

In another embodiment, the present invention provides a process for the preparation of compound of formula (I):

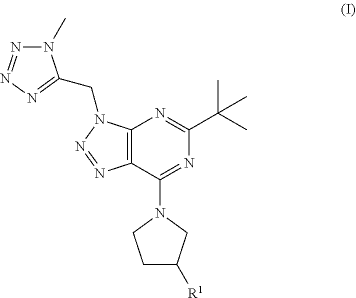

wherein R¹ is as herein defined,
   which comprises
a) reacting the compound of formula (IV)

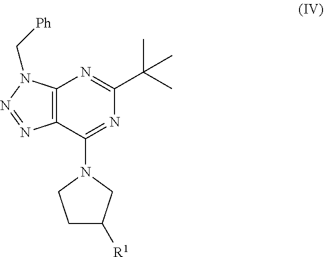

wherein R¹ is as herein defined,
with H₂, in particular in the presence of a suitable heterogeneous transition metal hydrogenation catalyst to produce a compound of formula (II), salts thereof, a tautomer thereof or a mixture of tautomers thereof, in particular in the presence of an organic acid;
b) reacting a compound of formula (II), salts thereof, a tautomer thereof or a mixture of tautomers thereof

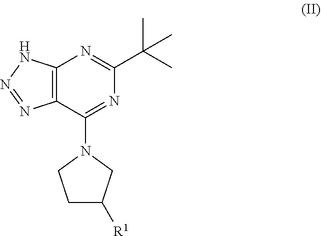

wherein R¹ is as herein defined,
with a compound of formula (III):

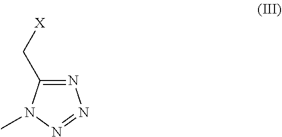

wherein X is as herein defined in the presence of an organic acid.

In another embodiment, the present invention provides a process for the preparation of compound of formula (I):

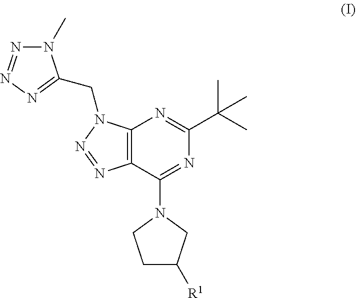

wherein R¹ is as herein defined,
   which comprises
a) reacting the compound of formula (IV)

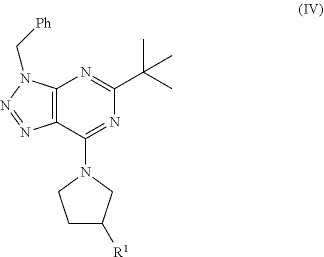

wherein R¹ is as herein defined,
with H₂ in particular in the presence of a suitable heterogeneous transition metal hydrogenation catalyst to produce a compound of formula II;
b) reacting a compound of formula (II), salts thereof, a tautomer thereof or a mixture of tautomers thereof

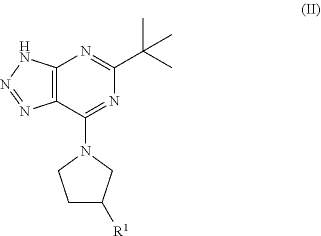

wherein R¹ is as herein defined,
with a compound of formula (III):

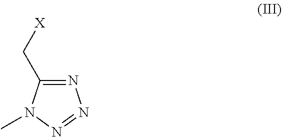

wherein X is as herein defined, in the presence of an organic acid during the workup phase.

In a more particular embodiment, the present invention provides a process as described herein for the preparation of compound of formula (I) or (I′) wherein the the heterogeneous transition metal hydrogenation catalyst is Raney catalyst (e.g. Ra-Ni, Ra-Co,) Pd/C, Pd(OH)2/C, Au/TiO₂, Rh/C, Ru/Al₂O₃, Ir/CaCO₃, or Pt/C, in particular Pd/C.

In a more particular embodiment, the present invention provides a process as described herein for the preparation of compound of formula (I) or (I′) with H₂ in the presence of an inorganic acid, in particular hydrochloric acid.

The present invention provides a process for the preparation of compound of formula (I):

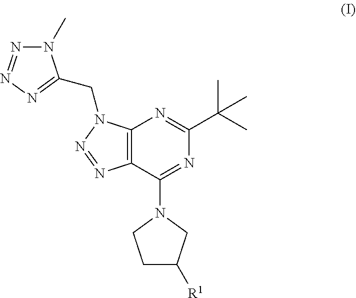

wherein
R¹ is halogen, —OH, —NR^{a}R^{b}, C₁₋₆ alkoxy, (C₁-C₆)alkyl, —O(O)CR^{c} or —NR^{a}C(O)R^{b};
R^{a} and R^{b} are chosen independently from H, (C₁-C₆)alkyl, C₁₋₆ alkoxy, haloalkyl, phenyl, halophenyl or alkylphenyl or a combination of thereof;
R^{c} is chosen independently from H, (C₁-C₆)alkyl, (C₁-C₆)alkoxy, —OH, C₁₋₆ halo-(C₁-C₆)alkyl, phenyl, halo-phenyl or (C₁-C₆)alkyl-phenyl;
or a pharmaceutically acceptable salt;
which comprises reacting a compound of formula (II), salts thereof, a tautomer thereof or a mixture of tautomers thereof

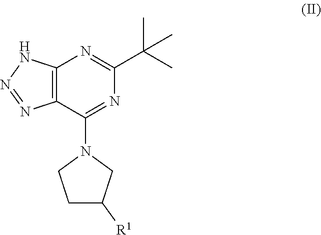

with a compound of formula (III):

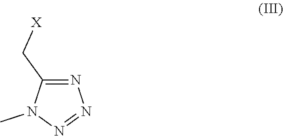

wherein
X is halogen, triflate or tosyl;
in the presence of an organic acid.

In a particular embodiment, the invention provides a process as described herein, wherein R¹ is —OH.

In a particular embodiment, the invention provides a process of making the compound of formula (I′) by reacting compound of formula (II′), a salt thereof, a tautomer thereof or a mixture of tautomers thereof with compound of formula (III′), in particular in the presence of an organic acid as defined herein. (scheme 1)

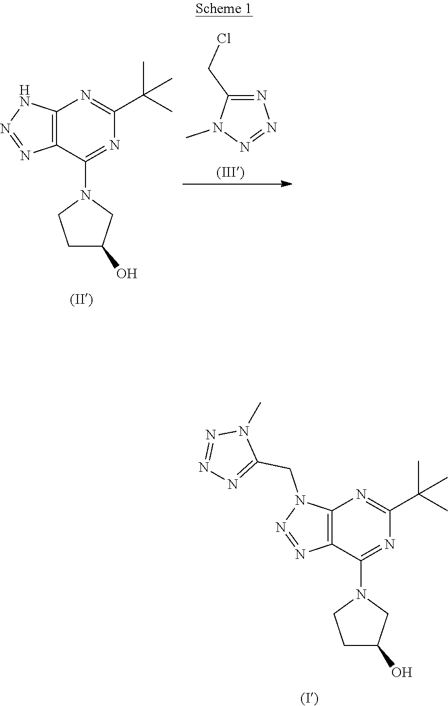

In another embodiment, the present invention provides a process for the preparation of compound of formula (I):

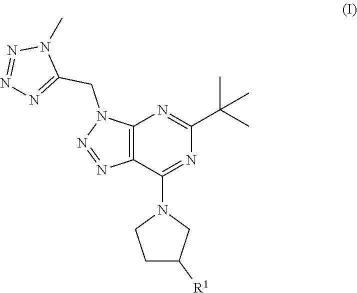

wherein R¹ is as herein defined,
   which comprises:
a) reacting the compound of formula (IV)

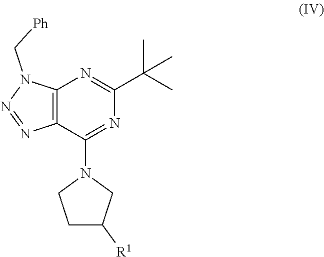

wherein R¹ is as herein defined,
with H₂ in particular in the presence of a suitable heterogeneous transition metal hydrogenation catalyst to produce a compound of formula (II), salts thereof, a tautomer thereof or a mixture of tautomers thereof;
b) reacting a compound of formula (II), salts thereof, a tautomer thereof or a mixture of tautomers thereof

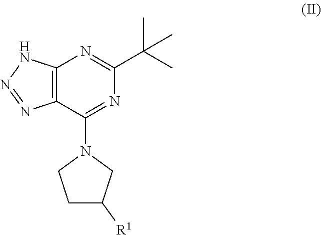

with a compound of formula (III):

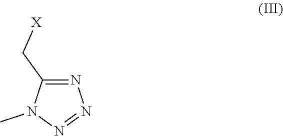

wherein X is as herein defined, in the presence of an organic acid.

In a particular embodiment, the invention provides a process of making a compound of formula (II′), a salt thereof, a tautomer thereof or a mixture of tautomers thereof from a compound of formula (IV′) comprising a reduction. (scheme 2)

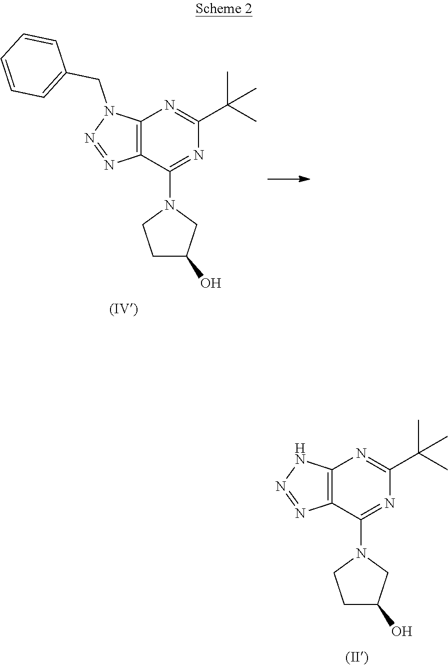 [SMILES: CC(C)(C)c1nc(N2CC[C@H](O)C2)c2nnn(Cc3ccccc3)c2n1 ; CC(C)(C)c1nc(N2CC[C@H](O)C2)c2nn[nH]c2n1]

The present invention provides a process of making the compounds of formulae (I′) and (Ia) by reacting a compound of formula (II′), a salt thereof, a tautomer thereof or a mixture of tautomers thereof with a compound of formula (III′) (Scheme 3).

In a particular embodiment, the invention provides the purification of compound of formula (I′) with an acidic workup in order to remove the undesired regioisomer compound of formula (Ia). It was surprisingly found that the use of an acidic extraction, performed using an organic acid, in particular an acidic organic solution, within a specific pH range enables the separation of the undesired isomer.

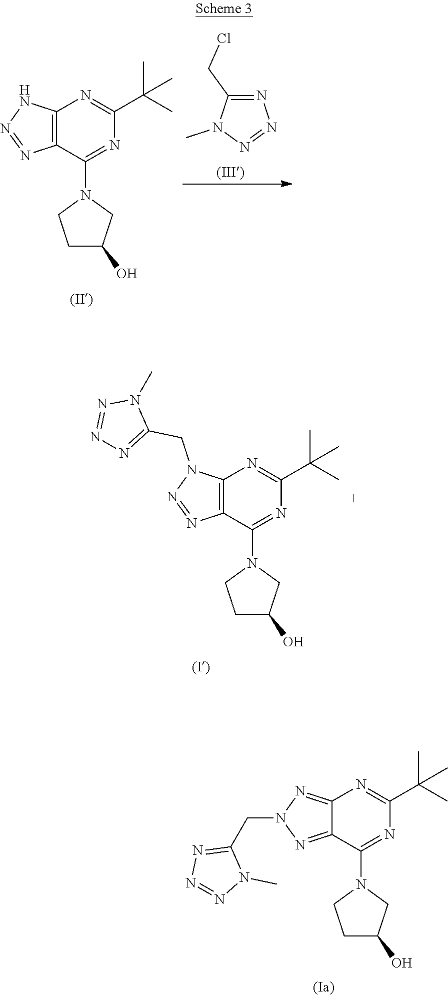

In another embodiment, the present invention provides a process for the preparation of compound of formula (I′):

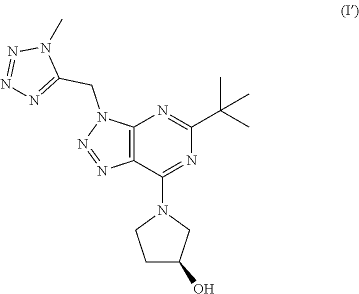 [SMILES: Cn1nnnc1Cn1nnc2c(N3CC[C@H](O)C3)nc(C(C)(C)C)nc21]

which comprises:
   a) reacting the compound of formula (IV′)

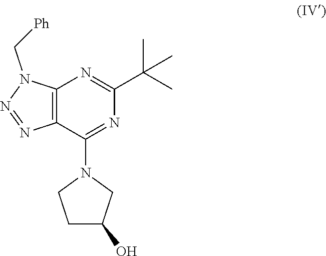 [SMILES: CC(C)(C)c1nc(N2CC[C@H](O)C2)c2nnn(Cc3ccccc3)c2n1]

with H₂ in particular in the presence of a suitable heterogeneous transition metal hydrogenation catalyst to produce a compound of formula (II′), a salt thereof, a tautomer thereof or a mixture of tautomers thereof
b) reacting a compound of formula (II′), a salt thereof, a tautomer thereof or a mixture of tautomers thereof

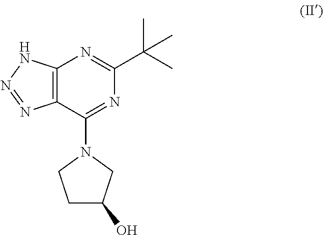 [SMILES: CC(C)(C)c1nc(N2CC[C@H](O)C2)c2nn[nH]c2n1]

with a compound of formula (III):

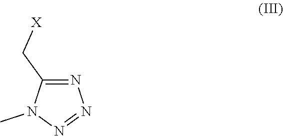

wherein X is as herein defined in the presence of an organic acid.
c) a recrystallization step of compound of formula (I′) with an appropriate solvent.
d) a jet milling process of compound of formula (I′)

In a particular embodiment, the invention provides a multistep synthetic route comprise of 4 steps as shown in scheme 4.

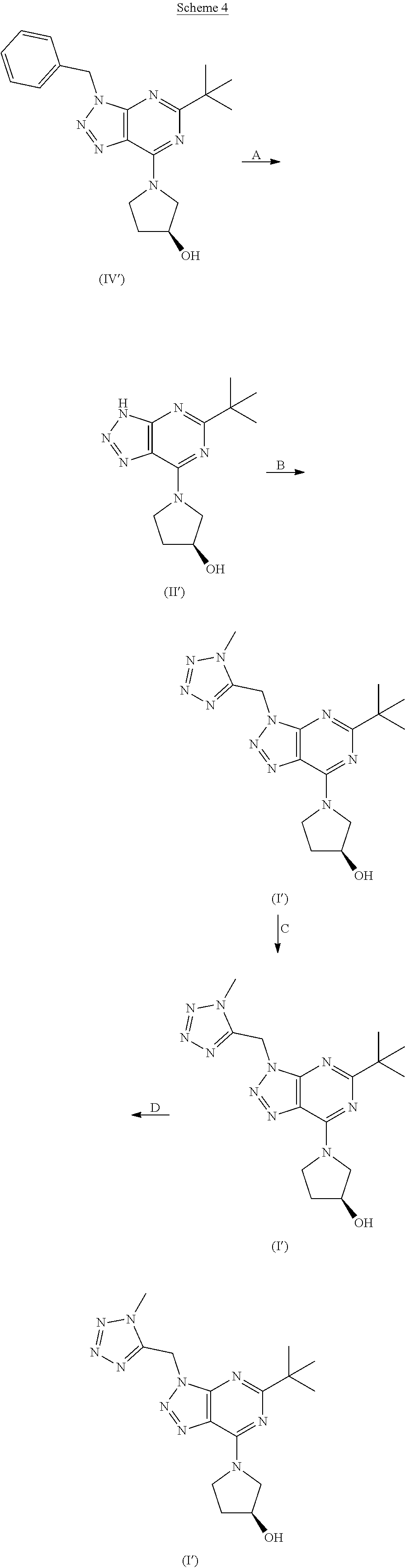

Wherein C is a recrystallization step of compound of formula (I′) with an appropriate solvent (e.g. isoamyl alcohol, iPrOAc/pentane, etc.), and step D is a jet milling process of compound of formula (I′).

In a particular embodiment the application further discloses a process of making compound of formula (IV′) from compound of formula (V) according to scheme 5.

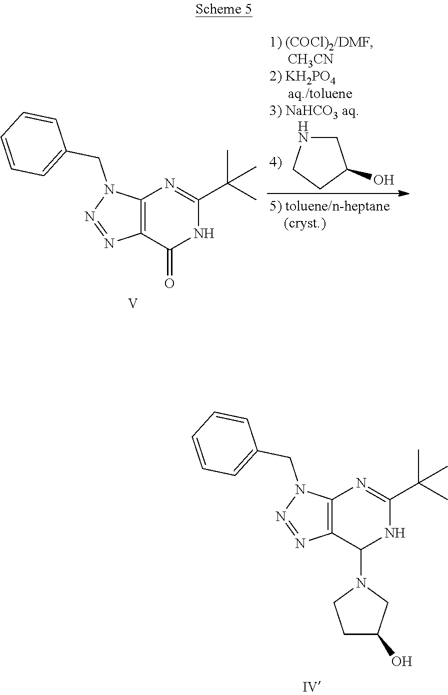

The starting materials, reagents and catalysts, which do not have their synthetic route explicitly disclosed herein, are generally available from commercial sources or are readily prepared using methods known to the person skilled in the art. For instance, the compounds of formulae (V) and (IV) can be prepared according to the procedures described in WO2013/068306.

The present invention provides a solid form A of compound of formula (I′) that is characterized by an IR spectrum comprising peaks: 1132 cm⁻¹, 1092 cm⁻¹, 1071 cm⁻¹±2 cm⁻¹.

In a particular embodiment, the invention provides a solid form A of compound of formula (I′) having peaks at position mentioned in Table 1.

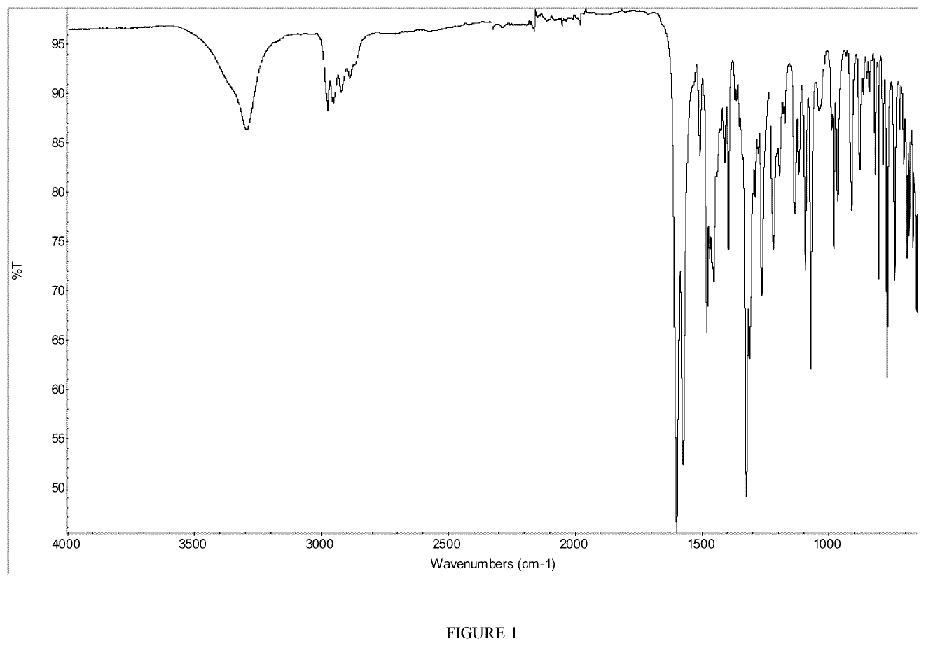

In a particular embodiment, the invention provides a solid form A of compound of formula (I′) having peaks at position according to FIG. 1.

TABLE 1
Infrared peak positions for Form A. The peak positions are
stated in cm⁻¹ and the error is ±2 cm⁻¹.

3295
2974
2953
2922
1599
1575
1508
1480
1469
1453
1410
1395
1364
1325
1312
1291
1278
1263
1217
1195
1173
1132
1119
1092
1071
1040
989
981
964
910
877
865
850
840
818
805
787
770
741
720
705
693
683
668
653

In a particular embodiment, the invention provides an IR spectrum of form A of compound of formula (I′). The ATR FTIR spectra were recorded without any sample preparation using a ThermoNicolet iS5 FTIR spectrometer with ATR accessory. The spectral range is between 4000 cm⁻¹ and 650 cm⁻¹, resolution 2 cm⁻¹ and 50 co-added scans were collected. Happ-Genzel apodization was applied. Using ATR FTIR will cause the relative intensities of infrared bands to differ from those seen in a transmission FTIR spectrum using KBr disc or nujol mull sample preparations. Due to the nature of ATR FTIR, the bands at lower wavenumber are more intense than those at higher wavenumber.

Peakpicking was performed using Thermo Scientific Omnic 8.3 software using the automated ‘Find Peaks’ function. The ‘threshold’ and ‘sensitivity’ were manually adjusted to get a representative number of peaks.

The present invention provides a solid form A of compound of formula (I′) that is characterized by a Raman spectrum comprising peaks: 1600 cm⁻¹, 1573 cm⁻¹, 1313 cm⁻¹±2 cm⁻¹. In a particular embodiment, the invention provides a Raman spectrum of form A of compound of formula (I′) with Raman peaks at positions as denoted in Table 2.

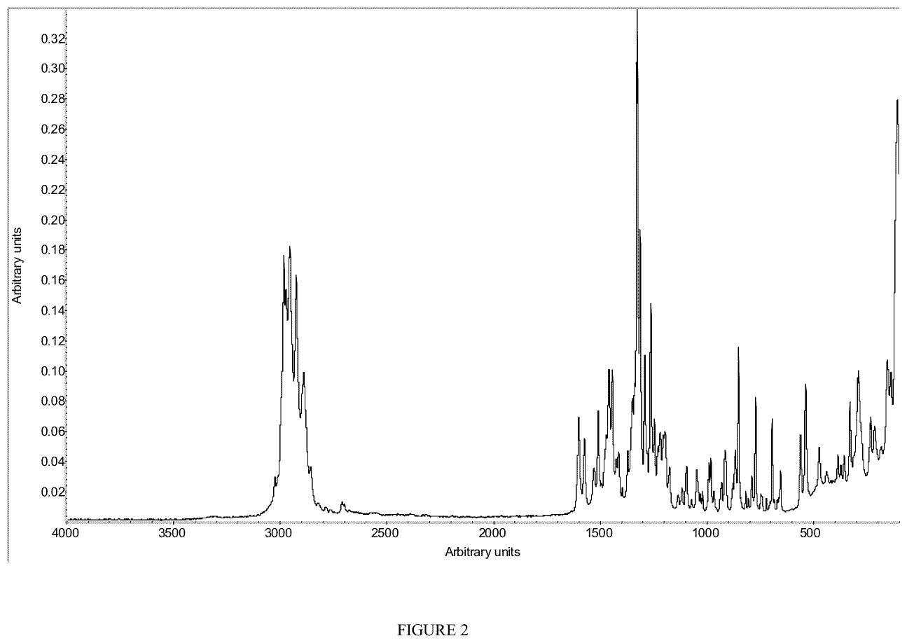

In a particular embodiment, the invention provides a solid form A of compound of formula (I′) having peaks at position according to FIG. 2.

TABLE 2
Raman peak positions for Form A. The peak positions are
stated in cm⁻¹ and the error is ±2 cm⁻¹.

2983
2954
2924
2889
1600
1573
1529
1509
1459
1444
1415
1371
1327
1313
1291
1263
1246
1218
1196
1174
1095
1048
990
981
931
916
865
852
788
772
694
654
561
537
473
386
356
329
291
232
214
153
138
107

In a particular embodiment, the invention provides a Raman spectrum of form A of compound of formula (I′). The FT-Raman spectrum was recorded without any sample preparation using a Bruker MultiRam FT-Raman spectrometer equipped with a liq N₂ cooled Germanium detector and 1064 nm NdYAG laser. The spectral range is between 4000 cm⁻¹ and 100 cm⁻¹, resolution 2 cm⁻¹ and 2048 co-added scans were collected. The laser power was set to 300 mW and Blackman-Harris 4-Term apodization was applied.

Peakpicking was performed using Thermo Scientific Omnic 8.3 software using the automated ‘Find Peaks’ function. The ‘threshold’ and ‘sensitivity’ were manually adjusted to get a representative number of peaks.

The present invention provides a solid amorphous form of compound of formula (I′) that is characterized by an IR spectrum comprising peaks: 1145 cm⁻¹, 1098 cm⁻¹, 918 cm⁻¹±2 cm⁻¹.

In a particular embodiment, the invention provides an IR spectrum of the amorphous form of compound of formula (I′) with the following peaks.

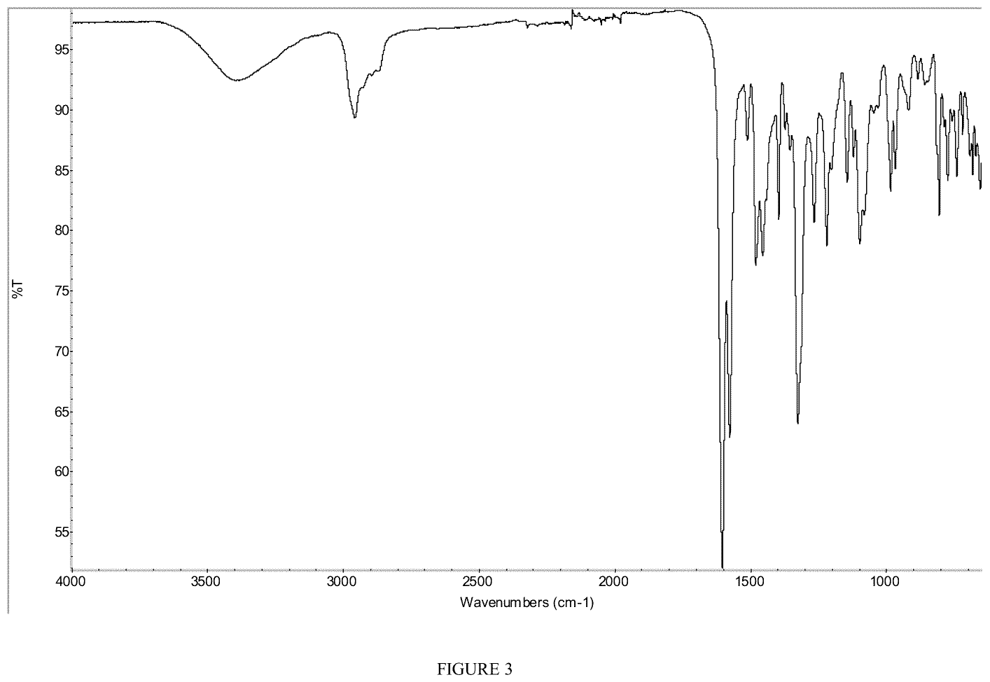

In a particular embodiment, the invention provides the amorphous form of compound of formula (I′) having peaks at position according to FIG. 3.

TABLE 3
Infrared peak positions for the amorphous form. The peak positions
are stated in cm⁻¹ and the error is ±2 cm⁻¹.

3394
2957
1604
1577
1512
1481
1455
1396
1373
1355
1326
1266
1219
1145
1121
1098
984
967
918
884
857
805
774
740
719
692
682
671
654

In a particular embodiment, the invention provides an IR spectrum of the amorphous form of compound of formula (I′). The ATR FTIR spectra were recorded without any sample preparation using a ThermoNicolet iS5 FTIR spectrometer with ATR accessory. The spectral range is between 4000 cm⁻¹ and 650 cm⁻¹, resolution 2 cm⁻¹ and 50 co-added scans were collected. Happ-Genzel apodization was applied. Using ATR FTIR will cause the relative intensities of infrared bands to differ from those seen in a transmission FTIR spectrum using KBr disc or nujol mull sample preparations. Due to the nature of ATR FTIR, the bands at lower wavenumber are more intense than those at higher wavenumber.

Peakpicking was performed using Thermo Scientific Omnic 8.3 software using the automated ‘Find Peaks’ function. The ‘threshold’ and ‘sensitivity’ were manually adjusted to get a representative number of peaks.

The present invention provides a solid amorphous form of compound of formula (I′) that is characterized by a Raman spectrum comprising peaks: 2961, 1607 cm⁻¹, 1514 cm⁻¹±2 cm⁻¹. In a particular embodiment, the invention provides a Raman spectrum of the amorphous form of compound of formula (I′) with the following peaks.

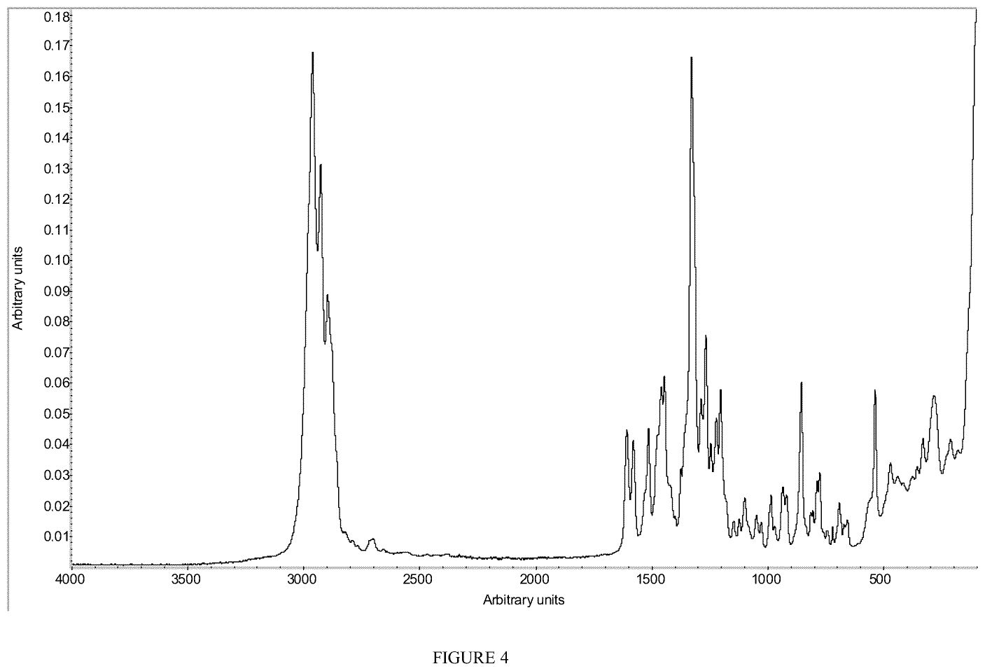

In a particular embodiment, the invention provides the amorphous form of compound of formula (I′) having peaks at position according to FIG. 4.

TABLE 4
Raman peak positions for the amorphous. The peak positions
are stated in cm⁻¹ and the error is ±2 cm⁻¹

2961
2926
2896
1607
1579
1514
1458
1445
1328
1287
1267
1245
1221
1203
1146
1122
1099
1048
1028
985
934
919
854
805
788
776
720
692
656
537
471
330
284
212

In a particular embodiment, the invention provides a Raman spectrum of the amorphous form of compound of formula (I′). The FT-Raman spectrum was recorded without any sample preparation using a Bruker MultiRam FT-Raman spectrometer equipped with a liq N₂ cooled Germanium detector and 1064 nm NdYAG laser. The spectral range is between 4000 cm⁻¹ and 100 cm⁻¹, resolution 2 cm⁻¹ and 2048 co-added scans were collected. The laser power was set to 300 mW and Blackman-Harris 4-Term apodization was applied.

Peakpicking was performed using Thermo Scientific Omnic 8.3 software using the automated ‘Find Peaks’ function. The ‘threshold’ and ‘sensitivity’ were manually adjusted to get a representative number of peaks.

In a particular embodiment, the invention provides x-ray powder diffraction spectrum of the amorphous form of compound of formula (I′). The x-ray diffraction patterns are recorded at ambient conditions in transmission geometry with a STOE STADI P diffractometer (Cu Kα radiation, primary Ge-monochromator, Mythen 1K silicon strip detector, angular range 3° to 42° 2Theta, 20 seconds measurement time per step). The samples are prepared and analyzed without further processing (e.g. grinding or sieving) of the substance.

As used herein, unless stated otherwise, the XRPD measurements are taken using copper Kα radiation wavelength 1.54187 A. XRPD peaks reported herein are measured using Cu Kα radiation, X=1.54187 A, typically at a temperature of 25±3° C.

Measurement and evaluation of the X-ray diffraction data is done using WinXPOW software (STOE & Cie GmbH, Darmstadt, Germany).

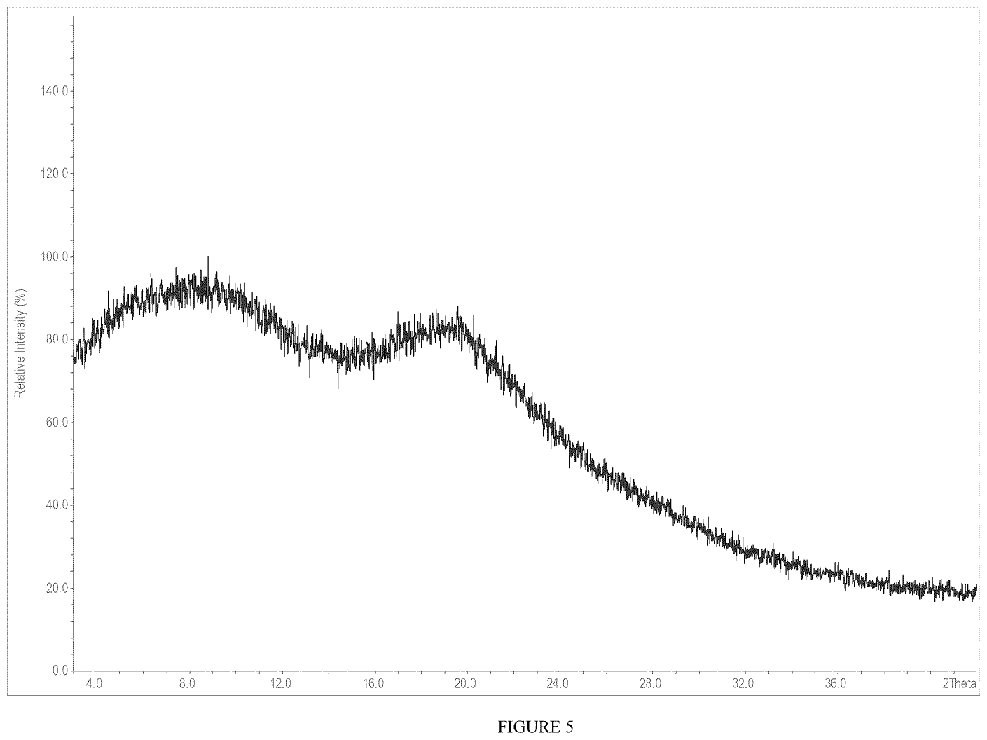

In a particular embodiment, the invention provides a solid form A of compound of formula (I′) having peaks at position according to FIG. 5.

The present invention provides a solid form A of compound of formula (I′) characterized by an X-ray powder diffraction pattern (XRPD) having characteristic peaks at an angle of diffraction 2-theta at about 9.88, 11.54, 16.01, 16.26, 18.17, and 20.31.

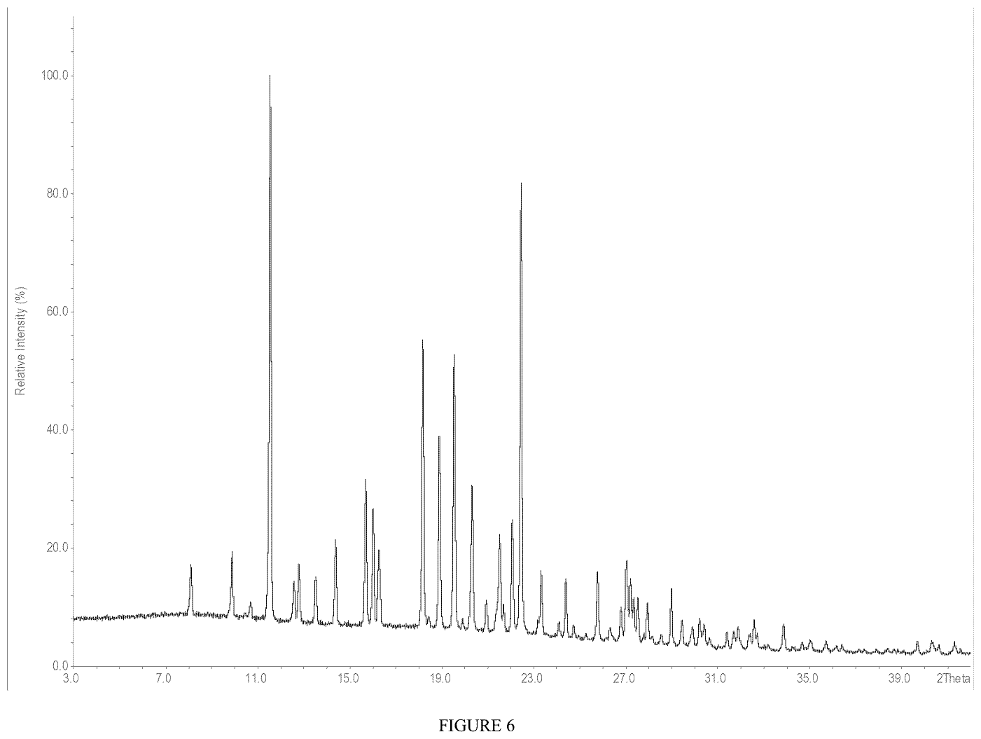

In a particular embodiment, the invention provides a solid form A of compound of formula (I′) having peaks at position according to FIG. 6.

In particular embodiment, Form A is characterized by XRPD diffraction pattern of comprising XRPD peaks at angles of diffraction 2-theta of as denoted in Table 5.

TABLE 5
XRPD of form A
Form A

| Peak position [° 2Theta] | Intensity [%] |
|---|---|
| 8.10 | 9 |
| 9.88 | 12 |
| 10.68 | 3 |
| 11.54 | 100 |
| 12.57 | 7 |
| 12.79 | 11 |
| 13.51 | 9 |
| 14.38 | 16 |
| 15.69 | 27 |
| 16.01 | 22 |
| 16.26 | 13 |
| 18.17 | 54 |
| 18.89 | 36 |
| 19.53 | 51 |
| 20.31 | 27 |
| 20.93 | 6 |
| 21.52 | 18 |
| 21.69 | 5 |
| 22.07 | 21 |
| 22.45 | 85 |
| 23.32 | 12 |
| 24.40 | 11 |
| 25.77 | 13 |
| 26.79 | 6 |
| 27.03 | 15 |
| 27.20 | 12 |
| 27.34 | 7 |
| 27.52 | 8 |
| 27.94 | 8 |
| 28.98 | 11 |
| 29.44 | 5 |
| 29.89 | 4 |
| 30.20 | 5 |
| 30.41 | 4 |
| 31.88 | 4 |
| 32.57 | 6 |
| 33.86 | 5 |

In a particular embodiment, the invention provides x-ray powder diffraction spectrum of form A of compound of formula (I′). The x-ray diffraction patterns are recorded at ambient conditions in transmission geometry with a STOE STADI P diffractometer (Cu Ku radiation, primary Ge-monochromator, Mythen 1K silicon strip detector, angular range 3° to 42° 2Theta, 20 seconds measurement time per step). The samples are prepared and analyzed without further processing (e.g. grinding or sieving) of the substance.

Measurement and evaluation of the X-ray diffraction data is done using WinXPOW software (STOE & Cie GmbH, Darmstadt, Germany).

Table 6 lists the relevant crystal structure data of Form A. The lattice constants, unit cell volume and calculated density are based at ambient temperature data.

TABLE 6
Single Crystal Structural Data of Form A

| space group | Orthorhombic, P2(1)2(1)2(1) | |
|---|---|---|
| a, Å | 9.10350 | (10) |
| b, Å | 17.6282 | (2) |
| c, Å | 21.8328 | (3) |
| alpha = beta = gamma, deg | 90 | |
| V, Å³ | 3503.69 | (7) |
| Z | 8 | |
| d_{calc}, g cm⁻³ | 1.359 | |
| temperature, K | 100 | (2) |

| | |
|---|---|
| radiation (wavelength, Å) | 1.54178 |
| linear abs coef, mm⁻¹ | 0.776 |

The invention as described herein demonstrates an improvement of a reaction, in particular regarding the yield, the solvent consumption, selectivity, and workup.

### EXAMPLES

The following examples are provided for illustration of the invention. They should not be considered as limiting the scope of the invention, but merely as being representative thereof.

### Example 1: Synthesis of (3S)-1-(3-benzyl-5-tert-butyl-triazolo[4,5-d]pyrimidin-7-yl)pyrrolidin-3-ol

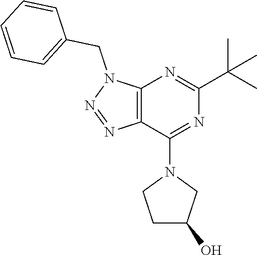 [SMILES: CC(C)(C)c1nc(N2CC[C@H](O)C2)c2nnn(Cc3ccccc3)c2n1]

3-benzyl-5-tert-butyl-6H-triazolo[4,5-d]pyrimidin-7-one (47.0 kg, 1.0 eq) was suspended in acetonitrile (321 kg) and N,N-dimethylformamide (30.3 kg, 2.5 eq) was added. Oxalyl chloride (42.1 kg, 2.0 eq) was added within 30 min at 35° C. and the mixture was then aged at 35° C. After complete conversion, the reaction mixture was added to a biphasic mixture of toluene (205 kg) and 8% aq. KH₂PO₄-solution (281 kg). After phase separation, the organic layer was washed with 5% aq. NaHCO₃ solution (282 kg). The organic phase was concentrated under reduced pressure and stripped with toluene in order to remove acetonitrile and residual water. N,N-Diisopropylethylamine (27.9 kg, 1.3 eq) was added to the solution at 20° C. followed by a solution of (S)-3-hydroxypyrrolidine (15.5 kg, 1.07 eq) in Ethanol (74 kg). The resulting reaction mixture was stirred at ambient temperature. After complete conversion, the organic phase was washed with water (140 kg) and the phases were separated. The organic phase was concentrated and n-Heptane (372 kg) was added at min. 55° C. The solution was seeded at 45° C., aged for 1 h, cooled to 0° C. and aged for 4 h. The solids were filtered off, washed and dried at 55° C. under reduced pressure. 50.0 kg of product were obtained.

### Example 2: Synthesis of (3S)-1-[5-tert-butyl-3-[(1-methyltetrazol-5-yl)methyl]triazolo[4,5-d]pyrimidin-7-yl]pyrrolidin-3-ol

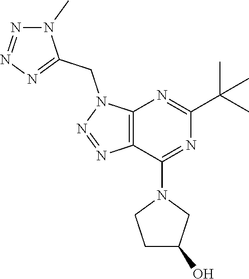 [SMILES: Cn1nnnc1Cn1nnc2c(N3CC[C@H](O)C3)nc(C(C)(C)C)nc21]

(3S)-1-(3-benzyl-5-tert-butyl-6,7-dihydrotriazolo[4,5-d]pyrimidin-7-yl)pyrrolidin-3-ol (89.0 kg 1.0 eq) was dissolved in 1-Propanol (356 kg). 10% Pd/C E 101 NE/W (9.6 kg as is, 4.43 kg dry), water (215 kg) and HCl 37% (29.9 kg, 1.2 eq.) were added and the suspension was hydrogenated at 60° C. and 8 barg H₂ for 4 h. 10% Pd/C E 101 NE/W (4.8 kg as is, 2.21 kg dry) was then added and the suspension was further hydrogenated for 14-25 h. Upon complete conversion, the mixture was cooled to 20° C. and pressure was released. The mixture was filtered and the reactor and filter cake were washed with water (366 kg).

To the filtrate, NaOH 28% (87 kg, 2.4 eq) was added and 1-Propanol was removed by distillation under reduced pressure. 5-(chloromethyl)-1-methyltetrazole (35.2 kg, 1.05 eq), n-Bu₄NI (9.3 kg, 0.10 eq) and n-Propylacetate (185 kg) were added. The biphasic mixture was warmed to 45° C. and stirred for 4 h. Upon complete conversion, the phases were settled and the aqueous layer was drained. The organic layer was extracted three times with a solution of Citric Acid aq. 10% (3×445 kg) followed by a wash with water (445 kg). The organic layer was filtered over ZetaCarbon and ZetaPlus modules and concentrated under reduced pressure. N-Heptane (138 kg) was added at 65-70° C. The solution was seeded, aged for 1 h and cooled to 0° C. and aged for 2 h. The solids were filtered, washed with n-Propylacetate/n-Heptane 2:1 (354 kg) and dried to obtain (3S)-1-(3-benzyl-5-tert-butyl-6,7-dihydrotriazolo[4,5-d]pyrimidin-7-yl)pyrrolidin-3-ol (45.0 kg) as a white solid.

### Example 3: Recrystallization of (3S)-1-[5-tert-butyl-3-[(1-methyltetrazol-5-yl)methyl]triazolo[4,5-d]pyrimidin-7-yl]pyrrolidin-3-ol

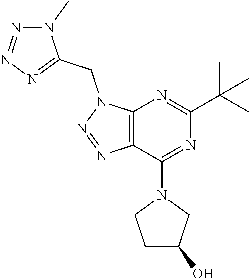 [SMILES: Cn1nnnc1Cn1nnc2c(N3CC[C@H](O)C3)nc(C(C)(C)C)nc21]

(3S)-1-[5-tert-butyl-3-[(1-methyltetrazol-5-yl)methyl]-6,7-dihydrotriazolo[4,5-d]pyrimidin-7-yl]pyrrolidin-3-ol crude (44.0 kg) was dissolved in Isoamyl alcohol (263 kg) at 75° C. The solution was filtered via a polish filter and the reactor was rinsed with Isoamyl alcohol (36 kg). The solution was cooled down to 54° C., seeded and aged for 4 h. The suspension was cooled down to 0° C., aged for 12 h and filtered. The wet cake was washed with Isoamyl alcohol/n-Heptane 3:2 (73 kg) and n-Heptane (73 kg). After drying at 50° C. under reduced pressure recrystallized (3S)-1-(3-benzyl-5-tert-butyl-6,7-dihydrotriazolo[4,5-d]pyrimidin-7-yl)pyrrolidin-3-ol was (36.5 kg) was obtained as a white solid. Recrystallized (3S)-1-(3-benzyl-5-tert-butyl-6,7-dihydrotriazolo[4,5-d]pyrimidin-7-yl)pyrrolidin-3-ol was further jet-milled with a fluidized bed opposed jet mill to obtain 35.0 kg of jet-milled (3S)-1-(3-benzyl-5-tert-butyl-6,7-dihydrotriazolo[4,5-d]pyrimidin-7-yl)pyrrolidin-3-ol.

1. A process for the preparation of compound of formula (I) or a pharmaceutically acceptable salt: 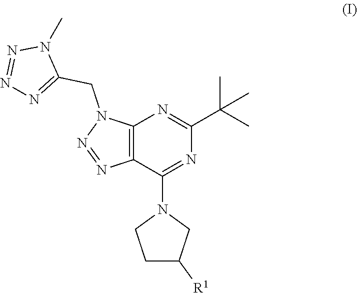
wherein
R¹ is halogen, —OH, —NR^{a}R^{b}, (C₁-C₆)alkoxy, (C₁-C₆)alkyl, —O(O)CR^{c} or —NR^{a}C(O)R^{b};
R^{a} and R^{b} are chosen independently from H, (C₁-C₆)alkyl, (C₁-C₆)alkoxy, C₁₋₆ halo-(C₁-C₆)alkyl, phenyl, halo-phenyl or (C₁-C₆)alkyl-phenyl;
R^{c} is chosen independently from H, (C₁-C₆)alkyl, (C₁-C₆)alkoxy, —OH, C₁₋₆ halo-(C₁-C₆)alkyl, phenyl, halo-phenyl or (C₁-C₆)alkyl-phenyl;
which comprises reacting a compound of formula (II), salts thereof, a tautomer thereof or a mixture of tautomers thereof 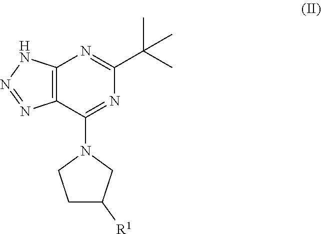
with a compound of formula (III): 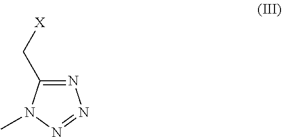
wherein
X is halogen, triflate or tosyl;
in the presence of an organic acid.

2. The process according to claim 1 for the preparation of compound of formula (I): 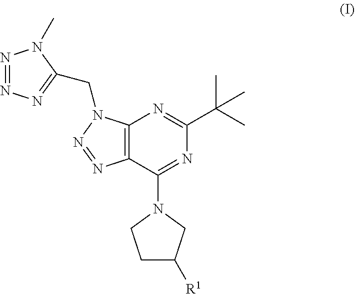
wherein R¹ is as defined according to claim 1
or a pharmaceutically acceptable salt;
which comprises
a) reacting the compound of formula (IV) 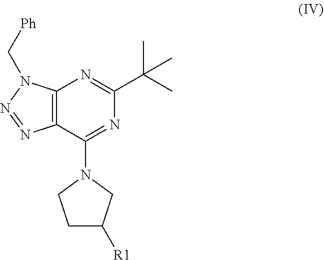
wherein R¹ is as defined according to claim 1,
with H₂, in particular in the presence of a suitable heterogeneous transition metal hydrogenation catalyst to produce a compound of formula (II), salts thereof, a tautomer thereof or a mixture of tautomers thereof;
b) reacting a compound of formula (II), salts thereof, a tautomer thereof or a mixture of tautomers thereof 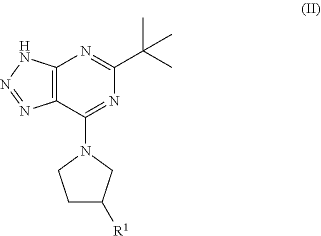
with a compound of formula (111): 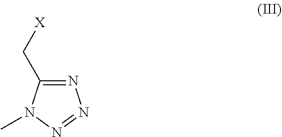
wherein X is as defined according to claim 1;
in the presence of an organic acid.

3. A process for the preparation of compound of formula (I′) or a pharmaceutically acceptable salt according to claim 1: 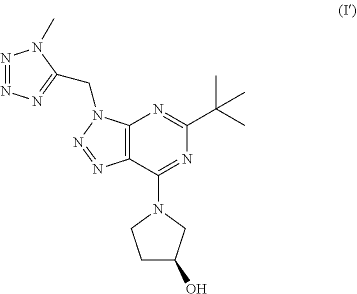 [SMILES: Cn1nnnc1Cn1nnc2c(N3CC[C@H](O)C3)nc(C(C)(C)C)nc21]
which comprises reacting a compound of formula (II′), or a salt thereof a tautomer thereof or a mixture of tautomers thereof 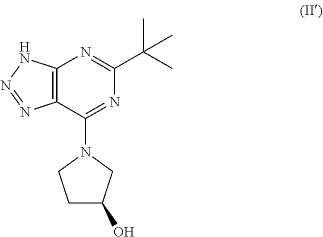 [SMILES: CC(C)(C)c1nc(N2CC[C@H](O)C2)c2nn[nH]c2n1]
with a compound of formula (III): 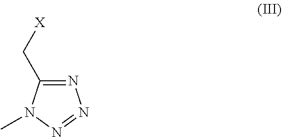
wherein
X is as defined according to claim 1;
in the presence of an organic acid.

4. The process according to any one of claim 3 for the preparation of compound of formula (I′) or a pharmaceutically acceptable salt 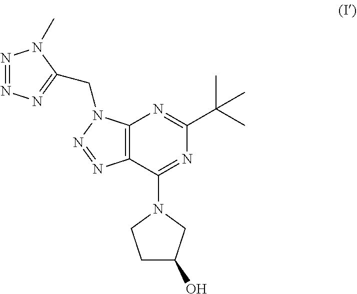 [SMILES: Cn1nnnc1Cn1nnc2c(N3CC[C@H](O)C3)nc(C(C)(C)C)nc21]
which comprises
a) reacting the compound of formula (IV′) 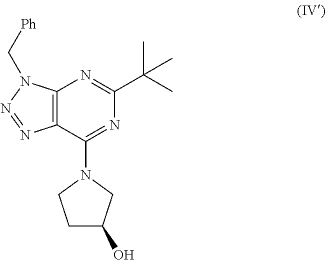 [SMILES: CC(C)(C)c1nc(N2CC[C@H](O)C2)c2nnn(Cc3ccccc3)c2n1]
with H₂, in particular in the presence of a suitable heterogeneous transition metal hydrogenation catalyst to produce a compound of formula (II′), salts thereof, a tautomer thereof or a mixture of tautomers thereof,
b) reacting a compound of formula (II′), salts thereof, a tautomer thereof or a mixture of tautomers thereof with a compound of formula (III), wherein X is as defined according to claim 1, in the presence of an organic acid.

5. The process of claim 1, wherein the compound of formula is a hydrochloric acid salt thereof.

6. The process according to claim 2, wherein the heterogeneous transition metal hydrogenation catalyst is Raney catalyst Pd/C, Pd(OH)₂/C, Au/TiO₂, Rh/C, Ru/Al₂O₃, Ir/CaCO₃, or Pt/C.

7. The process according to claim 2, in the presence of an inorganic acid.

8. The process according to claim 1, which further comprises a phase transfer catalyst.

9. The process according to claim 1, wherein the organic acid is in a suitable solvent, forming an acidic organic solution.

10. The process according to claim 9, wherein the suitable solvent is selected from water, methanol, or ethanol, particularly water.

11. The process according to claim 9, wherein the acidic organic solution has a pH between 1 and 4, particularly between 2 and 3, and more particularly around 2.5.

12. The process according to claim 1, wherein the organic acid is selected from lactic acid, formic acid, citric acid, oxalic acid, malic acid.

13. The process according to a claim 9, wherein the concentration of the acidic organic solution is between 1% and 30%, particularly between 5% and 20%, more particularly around 10%.

14. The process according to claim 1, in the presence of a biphasic solvent mixture.

15. The process according to claim 14, wherein the biphasic solvent mixture is between water and any of the solvents selected from ethyl acetate, diethyl carbonate, diethyl ether, methyl t-butyl ether, isopropyl acetate, n-propyl acetate, tetrahydrofuran, MeTHF, or a combination thereof.

16. The process according to claim 1, in the presence of an inorganic base.

17. The process according to claim 16, wherein the inorganic base is sodium carbonate, potassium carbonate, lithium carbonate, lithium hydroxide, potassium hydroxide, sodium hydroxide, sodium hydrogen carbonate, potassium hydrogen carbonate, or lithium hydrogen carbonate.

18. The process according to claim 8, wherein the phase transfer catalyst is selected from a quaternary ammonium salt, an organic phosphonium salt or a crown ether.

19. A solid form A of compound of formula (I) characterized by an X-ray powder diffraction pattern (XRPD) having characteristic peaks at an angle of diffraction 2-theta at about 9.88, 11.54, 16.01, 16.26, 18.17, and 20.31; wherein the XRPD measurements are taken using copper K a radiation wavelength 1.54187 A.

20. The solid form A according to claim 19, further characterized by an X-ray powder diffraction pattern (XRPD) having characteristic peaks at an angle of diffraction 2-theta at about: 8.10, 9.88, 10.68, 11.54, 12.57, 12.79, 13.51, 14.38, 15.69, 16.01, 16.26, 18.17, 18.89, 19.53, 20.31, 20.93, 21.52, 21.69, 22.07, 22.45, 23.32, 24.40, 25.77, 26.79, 27.03, 27.20, 27.34, 27.52, 27.94, 28.98, 29.44, 29.89, 30.20, 30.41, 31.88, 32.57, and 33.86; wherein the XRPD measurements are taken using copper K a radiation wavelength 1.54187 A.

21. The solid form A according to claim 19, further characterized by an IR spectrum comprising peaks: 1132 cm⁻¹, 1092 cm⁻¹, 1071 cm⁻¹±2 cm⁻¹.

22. A process for the preparation of compounds of formula (I′) and (Ia), or a pharmaceutically acceptable salt thereof, 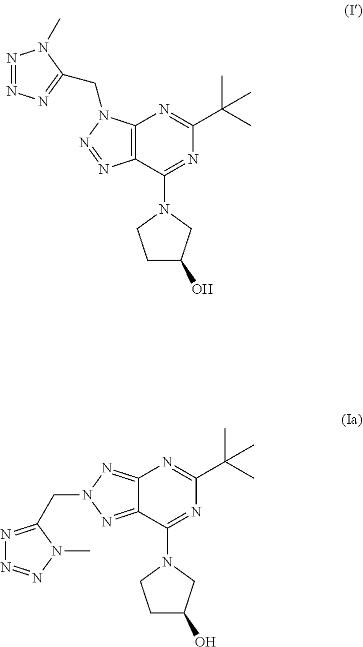 [SMILES: Cn1nnnc1Cn1nnc2c(N3CC[C@H](O)C3)nc(C(C)(C)C)nc21 ; Cn1nnnc1Cn1nc2nc(C(C)(C)C)nc(N3CC[C@H](O)C3)c2n1]
are produced by reacting a compound of formula (II′), a salt thereof, a tautomer thereof or a mixture of tautomers thereof 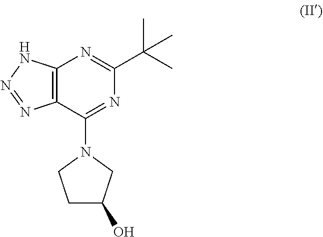 [SMILES: CC(C)(C)c1nc(N2CC[C@H](O)C2)c2nn[nH]c2n1]
with a compound of formula (III) 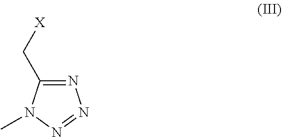
wherein
X is as defined according to claim 1.

23. The process according to claim 3, which further comprises the preparation of the compound for formula (IV′) 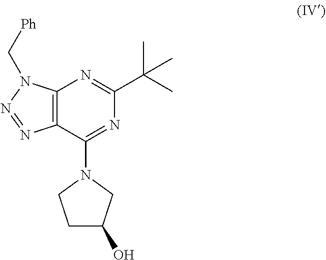 [SMILES: CC(C)(C)c1nc(N2CC[C@H](O)C2)c2nnn(Cc3ccccc3)c2n1]
by reacting the compound of formula (V) 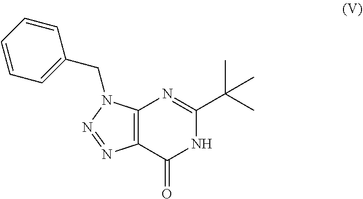 [SMILES: CC(C)(C)c1nc2c(nnn2Cc2ccccc2)c(=O)[nH]1]
with hydroxypyrrolidine.

24. (canceled)
